(19) United States
(12) Patent Application Publication
(10) Pub. No.: US 2026/0364488 A1
(43) Pub. Date: Nov. 19, 2026

(54) CHROMATOGRAPHY LIGAND AND CHROMATOGRAPHY MATERIAL, AND USES THEREOF

(71) Applicant: Cytiva BioProcess R&D AB, Uppsala (SE)

(72) Inventors: Jean-Luc Maloisel, Uppsala (SE); Gunnar Malmquist, Uppsala (SE); Lalita Shekhawat Kanwar, Uppsala (SE); Todd F. Markle, Uppsala (SE); Keyhan Esfandiarfard, Uppsala (SE)

(21) Appl. No.: 19/111,705

(22) PCT Filed: Sep. 27, 2023
(86) PCT No.: PCT/EP2023/076622

§ 371 Date: Mar. 13, 2025

(30) Foreign Application Priority Data
Sep. 29, 2022 (GB) 2214280.6

(51) Int. Cl. B01D 15/38; B01D 15/42; B01J 20/22; B01J 20/291; C07K 1/16

(52) U.S. Cl. CPC B01D 15/3809; B01D 15/424; B01J 20/22; B01J 20/291; C07K 1/16

(57) ABSTRACT

The present disclosure provides a chromatography ligand defined by Formula I: wherein X₁ is selected from CO and SO₂; each of R₁-R₅ is independently selected from H, F, Cl, O, N, S, C₁₋₃ alkyl, and C₁₋₃ alkyl-X₂; any two adjacent moieties selected from R₁-R₅ together with the atoms to which they are attached may form a 5- or 6-membered heterocyclic or carbocyclic ring; and X₂ is selected from O, S, NH(CO), (CO)NH, NH(SO₂), and (SO₂)NH; and provided that: i. when each of R₁-R₅ is H, X₁ is SO₂; ii. when any two adjacent moieties selected from R₁-R₅ together with the atoms to which they are attached form a 5-membered heterocyclic containing ring containing two oxygen atoms in the ring, X₁ is SO₂; and iii. when three of R₁-R₅ are CH₃O, X₁ is CO. The present disclosure also relates to a chromatography material comprising a support and the herein disclosed chromatography ligand coupled to the support. Further disclosed is a use of said chromatography material for separating one or more target molecules from impurities, as well as a method for separating one or more target molecules from impurities.

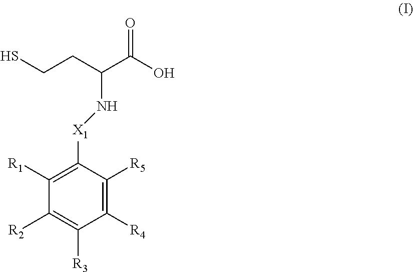

### FIELD OF THE DISCLOSURE

The present disclosure is directed to a chromatography ligand and a chromatography material, uses thereof for separating one or more target molecules from impurities, and a method for separating one or more target molecules from impurities. In particular, the present disclosure relates to separation of target molecules, such as antibodies or antibody fragments, from impurities comprising aggregates of the one or more target molecules.

### BACKGROUND OF THE DISCLOSURE

Bio-macromolecules, such as proteins, nucleic acids, and polysaccharides, may often partially occur in the form of aggregates, or multimers, such as dimers, trimers, or higher oligomers. Within the field of biological production of recombinant proteins, where desired polypeptides or proteins are produced in host organisms and isolated from cells or cell extracts under conditions and in concentrations quite different from those in their natural environment, the conditions may favor the formation of such aggregates through intermolecular disulphide linkages or other covalent bonds, or through non-covalent interactions. The presence of such aggregates of a target macromolecule is many times undesired. Protein aggregation is thus a common issue encountered during bioprocess development and manufacturing of biotherapeutics. Aggregated forms of a macromolecule may have lower biological activity than the non-aggregated form of the macromolecule; it may even completely lack the desired biological activity or may cause undesired side-effects. Hence, it is essential for therapeutic safety that a therapeutic protein is in a non-aggregated state and that there are no aggregates of molecules present in the final product.

Preparative chromatography remains the primary technique for purification of therapeutic proteins due to its benefits of resolution, scalability, and robustness. Monoclonal antibodies are one of the most powerful therapeutic tools in curing an increasing number of diseases. The continued development in upstream processing has resulted in increased purification complexities with regard to the types and content of impurities, e.g. due to higher titers. Also, the heterogeneity of monoclonal antibodies with differences in their charge distribution and size due to various molecular modifications over its lifespan from cell culture to polishing increases challenges in the polishing steps. The potential molecular modifications include charged acidic and basic variants, Fab fragment, and high molecular weight (HMW) aggregates, as well as glycosylation, deamidation, incomplete disulphide bond formation, oxidation, and isomerization etc. resulting in the formation of additional product related impurities. The biopharmaceutical industry is also facing new purification challenges due to increased molecular diversity of multi-specific antibodies with new and difficult product related impurities requiring more powerful polishing steps to attain the required product quality. With these increasing challenges, however, critical quality attributes remain high, increasing the demands on chromatography media for high resolution polishing.

Multimodal (or mixed mode) chromatography, where small molecule ligands provide more than one type of interaction, is an important tool in downstream processing of therapeutic proteins. A commercialized example of a multimodal is Capto™ MMC ImpRes (Cytiva Sweden AB, Uppsala, Sweden). It is a weak cation exchange multimodal ligand that enables high selectivity in a broad pH and salt window compared with traditional ion exchangers. It achieves efficient removal of aggregates, viruses, and main contaminants in processes for the purification of monoclonal antibodies and is suitable for polishing of antibody fragments.

However, there is a continuous need in the art for alternative chromatography ligands and use thereof in methods for improved separation of target molecules from impurities such as aggregates, such as high molecular weight aggregates of the target molecules.

### SUMMARY OF THE DISCLOSURE

The above objective to provide alternative chromatography ligands and methods for improved separation of target molecules from impurities is achieved by the present disclosure, which is directed to novel chromatography ligands and uses thereof.

More particularly, the presently disclosed chromatography ligand is defined by formula I:

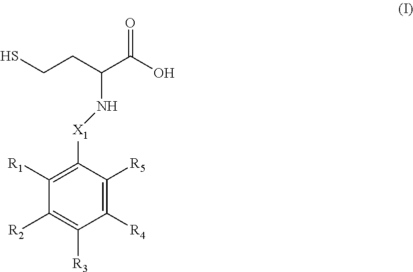

wherein:
X₁ is selected from CO and SO₂;
each of R₁-R₅ is independently selected from H, F, Cl, O, N, S, C₁₋₃ alkyl, and C₁₋₃ alkyl-X₂;
any two adjacent moieties selected from R₁-R₅ together with the atoms to which they are attached may form a 5- or 6-membered heterocyclic or carbocyclic ring; and
X₂ is selected from O, S, NH(CO), (CO)NH, NH(SO₂), and (SO₂)NH; and
provided that:
   i. when each of R₁-R₅ is H, X₁ is SO₂;
   ii. when any two adjacent moieties selected from R₁-R₅ together with the atoms to which they are attached form a 5-membered heterocyclic containing ring containing two oxygen atoms in the ring, X₁ is SO₂; and
   iii. when three of R₁-R₅ are CH₃O, X₁ is CO.

The present disclosure further provides a method for preparing a chromatography material, comprising immobilising a plurality of the above-defined chromatography ligand to a support.

Also provided is a chromatography material comprising the above-defined chromatography ligand coupled to a support.

Further provided is a use of the herein disclosed chromatography material for separating one or more target molecules from impurities.

The present disclosure also provides a method for separating one or more target molecules from impurities, comprising:
a) adding a liquid sample comprising one or more target molecules and impurities to a chromatography material as disclosed herein;
b) eluting the target molecules from the chromatography material;
c) optionally eluting the impurities from the chromatography material.

Also provided is a method for separating one or more target molecules from impurities, comprising:
a) adding a liquid sample comprising one or more target molecules and impurities to a chromatography material as disclosed herein;
b) obtaining the target molecules in a flow-through mode, the target molecules having passed through the chromatography material essentially without binding to the chromatography material;
c) optionally eluting the impurities from the chromatography material.

Preferred aspects of the present disclosure are described below in the detailed description and in the dependent claims.

### BRIEF DESCRIPTION OF THE DRAWINGS

FIG. 1 is a flow chart of a first method for separating one or more target molecules from impurities according to the present disclosure.

FIG. 2 is a flow chart of a second, alternative method for separating one or more target molecules from impurities according to the present disclosure.

FIG. 3 shows a comparison of separation resolution between HMW aggregate and monoclonal antibody (mAb) for different ligand prototypes for the data obtained using Method1 as described in Example 1 herein.

FIG. 4 shows a comparison of separation resolution between HMW aggregate and mAb for different ligand prototypes for the data obtained using Method2 as described in Example 1 herein.

FIG. 5 displays retention data for three of the novel ligand prototypes as described in Example 1 herein.

### DETAILED DESCRIPTION OF THE DISCLOSURE

The present disclosure is directed to chromatography ligands for improved separation of one or more target molecules from impurities, wherein the impurities may include aggregates and/or fragments of said target molecules.

As described in the Examples herein, the previously known, multimodal Capto MMC ImpRes ligand (Cytiva Sweden AB, Uppsala, Sweden) was chosen as a starting point for creating a large, chemically diverse virtual library of 100 Capto MMC analogue ligand structures. Based on these structures, physiochemical properties were predicted in silico, yielding a matrix of numerical descriptors (e.g. pKa, cLogP, etc). A subsequent principal component analysis (PCA) of these ligand descriptors resulted in a large chemical diversity map, which was used to select ligands for synthesis and coupling to an agarose base matrix. High-throughput plate-based screening, as well as analysis of column retention and resolution then generated numerical descriptors of chromatographic separation performance, which were connected to the chemical descriptors, guiding further cycles of synthesis and evaluation of the column separation resolution.

The present disclosure describes the selection, synthesis, and chromatographic evaluation of a smaller library of novel multimodal ligands against the reference ligand, i.e., Capto MMC ImpRes. It is shown in the Examples herein that the novel ligands, which are more hydrophobic than the reference ligand, achieved improved separation resolution between a monoclonal antibody and product-related impurities (i.e., aggregates and Fab fragments of said antibody), compared to the separation achieved by the reference ligand, when using linear salt gradient elution. More specifically, the monoclonal antibody was obtained at a higher purity and higher yield by use of the novel ligands. The prominent role of the secondary hydrophobic and hydrogen bonding interactions provided by the ligand chemical structure of more hydrophobic ligands in conjunction with electrostatic interactions results in their better performance with regard to removal of Fab fragment and aggregate impurities.

The present disclosure provides a chromatography ligand defined by formula I:

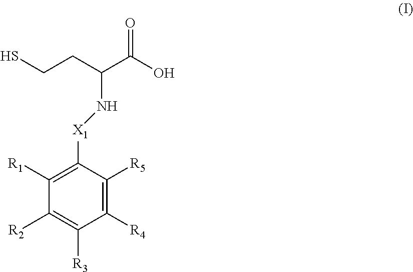

wherein:
X₁ is selected from CO and SO₂;
each of R₁-R₅ is independently selected from H, F, Cl, O, N, S, C₁₋₃ alkyl, and C₁₋₃ alkyl-X₂;
any two adjacent moieties selected from R₁-R₅ together with the atoms to which they are attached may form a 5- or 6-membered heterocyclic or carbocyclic ring; and
X₂ is selected from O, S, NH(CO), (CO)NH, NH(SO₂), and (SO₂)NH; and
provided that:
   i. when each of R₁-R₅ is H, X₁ is SO₂;
   ii. when any two adjacent moieties selected from R₁-R₅ together with the atoms to which they are attached form a 5-membered heterocyclic containing ring containing two oxygen atoms in the ring, X₁ is SO₂; and
   iii. when three of R₁-R₅ are CH₃O, X₁ is CO.

The 5- or 6-membered heterocyclic or carbocyclic ring may be unsaturated or saturated. Further, the 5- or 6-membered heterocyclic or carbocyclic ring may be non-polar, aromatic, and/or aliphatic. The heterocyclic ring may contain up to three heteroatoms. When there are three heteroatoms, all of them are N. When there are a maximum of two heteroatoms, each of them may be independently selected from N, O, and S.

In one embodiment, any two adjacent moieties selected from R₁-R₅, together with the atoms to which they are attached, may form a 5-membered heterocyclic ring containing a maximum of one oxygen atom in the ring.

When the chromatography ligand is coupled to a support, this may be illustrated according to the following (Formula Ia):

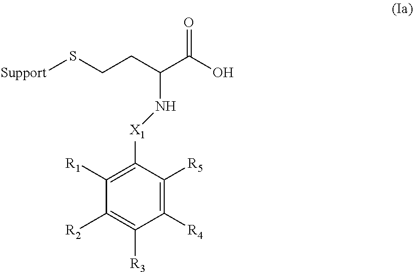

The “support” moiety of formula Ia represents a support, such as a chromatography bead, to which the ligand can be coupled. The ligand is connected to the support via a covalent thioether bond formed at the thiol.

Formula Ia (a chromatography ligand coupled to a support) may alternatively be depicted as

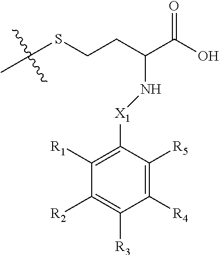

wherein the wavy moiety represents the coupling to said support. Hereinafter the term “support” is used in formula Ia to show where the ligand may bind to the support.

The term “chromatography ligand” means a molecule that has a known or unknown affinity for a given analyte and can be coupled to a support of a chromatography material, whereas “analyte” includes any specific binding partner to the ligand.

The analytes of interest to separate according to the present disclosure are so-called target molecules and impurities, which are present in a liquid sample.

In this context, the term “target molecule” is intended to include macromolecules which are to be separated from a liquid sample and purified from impurities before being put to use in their intended applications, for example as therapeutic substances.

The term “macromolecule” has its conventional meaning in the field of bioprocessing, in which macromolecules are produced (often recombinantly) by cells in a cell culture and purified from the cell culture by any means of separation and purification. Alternatively, the macromolecules are present in a biological solution which does not necessarily originate from a cell culture. Non-limiting examples of macromolecules are biomacromolecules, which are large biological polymers that are made up of monomers linked together, such as peptides and proteins (which can be native or recombinant), including but not limited to enzymes, antibodies and antibody fragments, as well as carbohydrates, and nucleic acid sequences, such as DNA and RNA. The macromolecule to be purified by use of the chromatography ligand according to the present disclosure is typically a protein or polypeptide, particularly a therapeutic protein or polypeptide, such as an antibody. Alternatively, the macromolecule may be a nucleic acid sequence, which may for example be used as a vector, such as in a therapeutic application. A macromolecule or a biomacromolecule may for example be a biopharmaceutical, i.e., a biological molecule, including but not limited to a biological macromolecule, which is intended for use as a pharmaceutical compound. It is to be understood that “a macromolecule” is intended to mean a type of macromolecule and that the singular form of the term may encompass a large number of individual macromolecules, or specimens, of the same type.

It is to be understood that the term “liquid sample” (or simply “sample”) as used herein encompasses any type of sample obtainable from a cell culture, or from a fluid originating from a cell culture which fluid is at least partly purified, by any means of separation and purification.

Herein, the term “cell culture” refers to a culture of cells or a group of cells being cultivated, wherein the cells may be any type of cells, such as bacterial cells, viral cells, fungal cells, insect cells, or mammalian cells. A cell culture may be unclarified, i.e., comprising cells, or may be cell-depleted, i.e., a culture comprising no or few cells but comprising biomolecules released from the cells before removing the cells. Further, an unclarified cell culture as used in the presently disclosed method may comprise intact cells, disrupted cells, a cell homogenate, and/or a cell lysate.

The term “antibody” as used herein means an immunoglobulin which may be natural or partly or wholly synthetically produced. The term includes, but is not limited to, whole (complete) antibodies, such as monospecific and multispecific antibodies. The term also includes active antibody fragments, including Fab antigen-binding fragments, univalent fragments, and bivalent fragments. The term also covers any protein having a binding domain which is homologous to an immunoglobulin binding domain. Such proteins can be derived from natural sources or be partly or wholly synthetically produced. The term further includes fusion proteins including an antibody or antibody fragment, e.g, monoclonal antibody or monoclonal antibody fragment covalently linked to other protein. Exemplary antibodies are the immunoglobulin isotypes and different types of fragments, such as Fab, Fab′, F (ab′)₂, Fv, dAb (single domain antibody), and Fd (fragment obtained by papain hydrolysis of an immunoglobulin molecule followed by reduction of the disulfide bonds), as well as scFv (so-called single-chain variable fragment, which is a fusion protein of the variable regions of the heavy and light chains of immunoglobulins), tandem scFvs, BiTEs (bispecific T-cell engager molecules), DARTs (dual-affinity retargeting molecules), and diabodies (single chain and tandem diabodies). A bispecific monoclonal antibody is an example of a multispecific antibody and is an artificial protein that can simultaneously bind to two different types of antigen or two different epitopes on the same antigen. The chromatography ligand according to the present disclosure may be used to purify for example a therapeutic antibody from impurities such as aggregates or fragments of said therapeutic antibody, to attain a high-quality end product. The presence of aggregates in therapeutic antibody preparations generally have a negative impact on patient safety and must be effectively removed during process manufacturing.

The term “vector” is herein used to denote a virus particle, normally a recombinant virus particle, which is intended for use to achieve gene transfer to modify specific cell type or tissue. A virus particle can for example be engineered to provide a vector expressing therapeutic genes. Several virus types are currently being investigated for use to deliver genetic material (e.g., genes) to cells to provide either transient or permanent transgene expression. These include adenoviruses, retroviruses (γ-retroviruses and lentiviruses), poxviruses, adeno-associated viruses (AAV), baculoviruses, and herpes simplex viruses.

A “virus particle” is herein used to denote a complete infectious virus particle. It includes a core, comprising the genome of the virus (i.e., the viral genome), either in the form of ribonucleic acid (RNA) or deoxyribonucleic acid (DNA), and the core is surrounded by a morphologically defined shell. The shell is called a capsid. The capsid and the enclosed viral genome together constitute the so-called nucleocapsid. The nucleocapsid of some viruses is surrounded by a lipoprotein bilayer envelope. In the field of bioprocessing, for the purpose of producing viral vectors for various applications such as therapy, the genome of a virus particle is modified to include a genetic insert, comprising genetic material of interest. Modified virus particles are allowed to infect host cells in a cell culture and the virus particles are propagated in said host cells, after which the virus particles are purified from the cell culture by any means of separation and purification.

Herein, the term “impurities” is intended to mean any molecule or substance which is present in the liquid sample, and which is not the desired target molecule. The term “impurities” includes aggregates, such as aggregates of the target molecule, such as high molecular weight aggregates of the target molecule. The term “impurities” further includes fragments of the target molecule, for example when the target molecule is an intact antibody and undesired fragments of said antibody are present in the liquid sample. The term “impurities” also includes host cell proteins (HCP), and in the case of bispecific antibodies, homodimers.

The target molecule according to the present disclosure is typically a non-aggregated macromolecule while the impurities, from which the target molecule is to be separated, typically include aggregates, and/or fragments, of said macromolecule, typically a protein, such as an antibody.

Herein, the term “non-aggregated macromolecule” is intended to mean a non-degraded macromolecule. A non-aggregated macromolecule may herein alternatively be called “non-degraded macromolecule” or “intact macromolecule”. In a typical embodiment herein, in which the macromolecule is a protein or a polypeptide, the non-aggregated macromolecule may be described as having an essentially intact tertiary structure, which usually involves an essentially hydrophilic surface of the macromolecule, while hydrophobic moieties are located in the interior of the macromolecule. Hence, a non-aggregated macromolecule essentially does not have hydrophobic moieties or hydrophobic groups exposed on the surface.

In contrast, in a protein or polypeptide which starts to degrade, the tertiary structure is gradually destroyed, which exposes hydrophobic moieties to the environment surrounding the protein or polypeptide. A protein or polypeptide macromolecule which is being degraded, or has been degraded, may form aggregates. A non-aggregated form of a macromolecule is in a monomeric state. Aggregates of a macromolecule may contain multimeric forms of the macromolecule, such as dimers, trimers etc. of the macromolecule. An individual macromolecule which is degrading may form aggregates with other individual, degrading, specimens of the same type of macromolecule, and/or may form aggregates with individual, degrading, specimens of other types of degrading macromolecules, or a combination thereof. Since aggregates of macromolecules contain degrading macromolecules, it follows that aggregates of macromolecules have hydrophobic moieties exposed on their surfaces.

So-called “high molecular weight (HMW) aggregates” is a term well-known to the skilled person. Such aggregates are formed by self-association of the target molecules (e.g., a monoclonal antibody having a molecular weight of approx. 150 kDa) with each other via covalent and non-covalent bonding. This results in the formation of dimers (e.g., approx. 300 kDa for monoclonal antibody dimers) or even higher order of aggregates, e.g., trimers (approx. 450 kDa for monoclonal antibody trimers). These aggregates can be either soluble or non-soluble based on the nature of the target molecule. Hence, the term “high molecular weight aggregate” may refer herein to an aggregate of a target molecule, which aggregate has a molecular weight which is approximately twice the molecular weight of the target molecule, or larger than twice the molecular weight of the target molecule.

Herein, the term “hydrophobic moiety” is intended to mean a hydrophobic part of the macromolecule or a hydrophobic group present in the macromolecule.

The term “hydrophobic group” as used herein is defined as a group of molecules which has a log P value >0. The partition coefficient, abbreviated P, is defined as a particular ratio of the concentrations of a solute between the two solvents (a biphase of liquid phases), specifically for un-ionized solutes, and the logarithm of the ratio is thus log P. When one of the solvents is water and the other is a non-polar solvent, then the log P value is a measure of lipophilicity or hydrophobicity. The defined precedent is for the lipophilic and hydrophilic phase types to always be in the numerator and denominator respectively; for example, in a biphasic system of n-octanol (hereafter simply “octanol”) and water:

$$log {P}_{oct / wat} = log ( \frac{{[ solute ]}_{octanol}^{un - ionized}}{{[ solute ]}_{water}^{un - ionized}} ) .$$

A log P value <0 indicates that a higher percentage of the solute is in the hydrophilic phase. Conversely, a log P value >0 indicates a higher percentage of the solute in the lipophilic phase, i.e., the hydrophobic phase.

As mentioned above, denatured macromolecules, as well as aggregates of a macromolecule are typically more hydrophobic than an intact, non-denatured, non-aggregated macromolecule. Aggregates therefore bind to a hydrophobic group of a chromatography ligand to a higher extent than a non-aggregated macromolecule. Aggregates of a target molecule also have a larger size than the target molecule as such, and thus have a larger surface area interacting with a chromatography ligand. Consequently, compared to a target molecule's binding to the presently disclosed multimodal ligand, aggregates of the target molecule will exhibit a stronger binding to the ligand. Thereby, in general an aggregate will elute later from a chromatography device than the target molecule.

On the other hand, a fragment of a target molecule is smaller in size than the intact target molecule and will therefore have a smaller surface interacting with a ligand. Further, for example a Fab fragment of a monoclonal antibody will in general mainly interact with a multimodal ligand via electrostatic interactions, while the intact monoclonal antibody generally will interact with the ligand via both hydrophobic and electrostatic interactions. Consequently, an intact monoclonal antibody in general exhibits a stronger binding to the presently disclosed multimodal ligand than a Fab fragment of the monoclonal antibody. Thereby, in general an intact antibody will elute later from a chromatography device than a fragment of the intact antibody.

In summary, the use of the presently disclosed chromatography ligand is based on utilizing differences in the ligand's binding to non-aggregated macromolecule and aggregates and/or fragments of the macromolecule, respectively.

As described further above, formula I is:

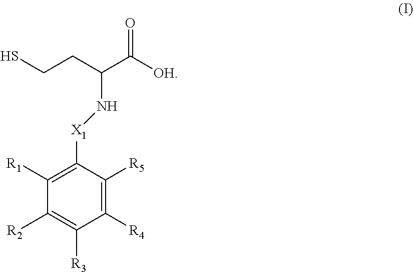

Formula I may be further defined by the following criteria:
X₁ is selected from CO and SO₂;
each of R₁-R₅ is independently selected from H, F, Cl, O, N, S, C₁₋₃ alkyl, and C₁₋₃ alkyl-X₂;
any two adjacent moieties selected from R₁-R₅ together with the atoms to which they are attached may form a 5- or 6-membered heterocyclic or carbocyclic ring; and
X₂ is selected from O, S, NH(CO), (CO)NH, NH(SO₂), and (SO₂)NH.

Formula I may be further defined by the following additional criteria:
i. when each of R₁-R₅ is H, X₁ is SO₂;
ii. when any two adjacent moieties selected from R₁-R₅ together with the atoms to which they are attached form a 5-membered heterocyclic containing ring containing two oxygen atoms in the ring, X₁ is SO₂; and
iii. when three of R₁-R₅ are CH₃O, X₁ is CO.

Further, the 5- or 6-membered heterocyclic or carbocyclic ring may be unsaturated or saturated. Further, the 5- or 6-membered heterocyclic or carbocyclic ring may be non-polar, aromatic, and/or aliphatic. The heterocyclic ring may contain up to three heteroatoms. When there are three heteroatoms, all of them are N. When there are a maximum of two heteroatoms, each of them may be independently selected from N, O, and S.

Herein, the expression “lysozyme batch binding capacity” is intended to mean the binding capacity of a chromatography material to a lysozyme in batch mode, wherein said chromatography material comprises a chromatography ligand coupled to a support as explained elsewhere herein.

Lysozyme was one of five model proteins used in a high-throughput plate-based study to test the presently disclosed chromatography ligands when present in said material, as described in detail in Example 1 below. More particularly, the binding capacity was studied by loading 60 μg of lysozyme on 6 μL of chromatography material (i.e., support coupled with the chromatography ligand) at binding conditions pH 7.5 and 480 mM NaCl in batch mode.

The term “adsorption isotherm” has its conventional meaning in the art. It describes the relationship between the equilibrium concentration of protein in solution and the amount bound on a chromatography ligand at a particular temperature and solution conditions like pH and ionic strength.

The reason to be in “the linear part of an adsorption isotherm” is to understand the selectivity between protein and ligand at different binding conditions of pH and ionic strength. It will also make the measurement of the amount of bound protein robust towards variations in the concentration of protein loaded onto the chromatography material.

The protein-ligand selectivity study for screening the pH and ionic strength window is never operated in the saturation part of the isotherm or overloaded conditions, as the selectivity between the protein-ligand is interfered by competitive binding of other impurities (as getting 100% pure monoclonal antibody for the study is not possible) and also protein-protein interactions comes into the picture at overloading conditions which is the characteristic feature of a non-linear isotherm. In the present study, the maximum limit for equilibrium binding capacity of lysozyme was 10 μg/μl of chromatography material (60 μg of lysozyme divided by 6 μl of chromatography material). The experimentally observed percentage equilibrium binding capacity can be calculated for each resin for each binding condition with respect to the maximum equilibrium binding capacity (10 μg/μL of resin). Here, a selection was made of those ligands which showed at least 50%, such as 60% of the maximum equilibrium binding capacity, i.e., the ligands which achieved an amount of bound lysozyme at equilibrium of at least 50%, such as 60% of the added amount of lysozyme.

The term “log S” has its conventional meaning in the art. Log S is directly related to the water solubility of a compound and it is defined as a common solubility unit corresponding to the 10-based logarithm of the solubility of a molecule measured in mol/L. log S is a measure of hydrophobicity; the more negative log S value, the more hydrophobic a chromatography ligand is.

The presently disclosed chromatography ligand may also further be defined by having a log S from about −2.5 to about −5. Log S values were calculated for the chromatography ligand structures with the support modeled as a methyl group. In other words, they were calculated for the methyl-S-ligand structures. More particularly, there is provided herein a chromatography ligand, wherein said ligand, when the —SH of formula I has been replaced with a methyl thioether (—S—CH₃), has a log S from about-2.5 to about −5.

More particularly, the presently disclosed chromatography ligand may be defined by a chemical structure selected from any one of (a)-(h):

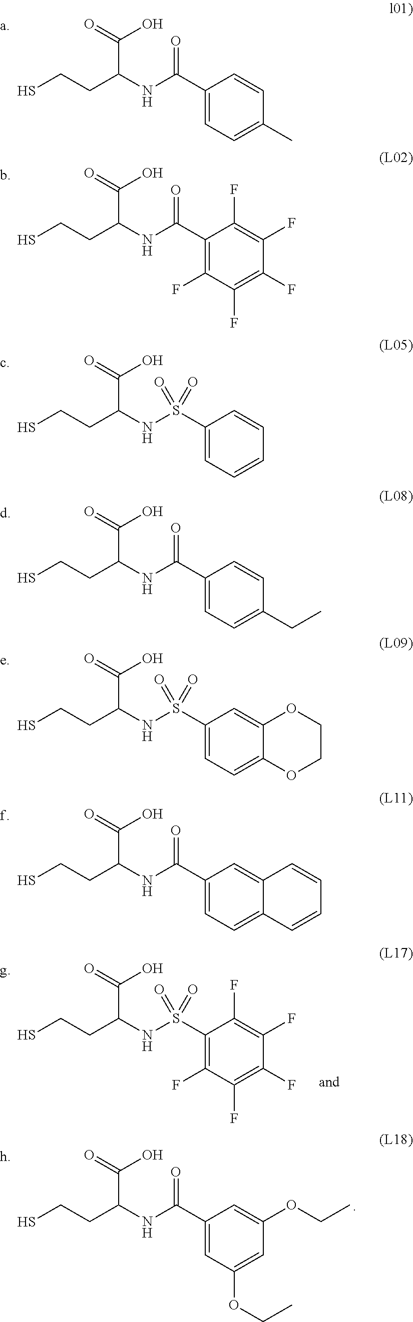

When the above chromatography ligands are coupled to a support, they may be illustrated as follows:

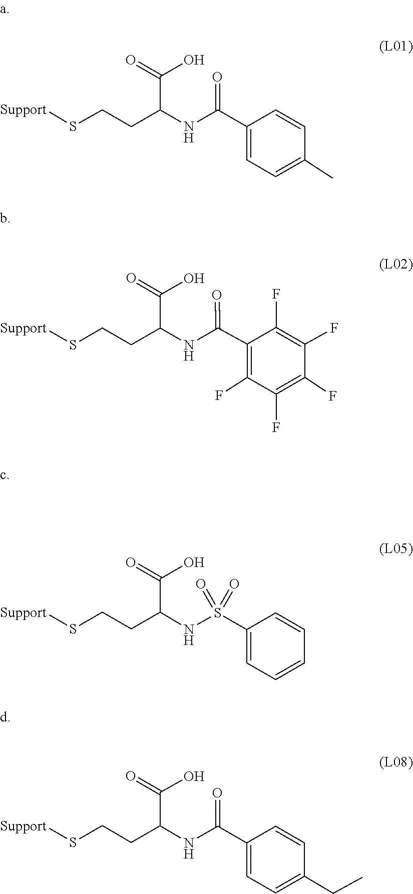

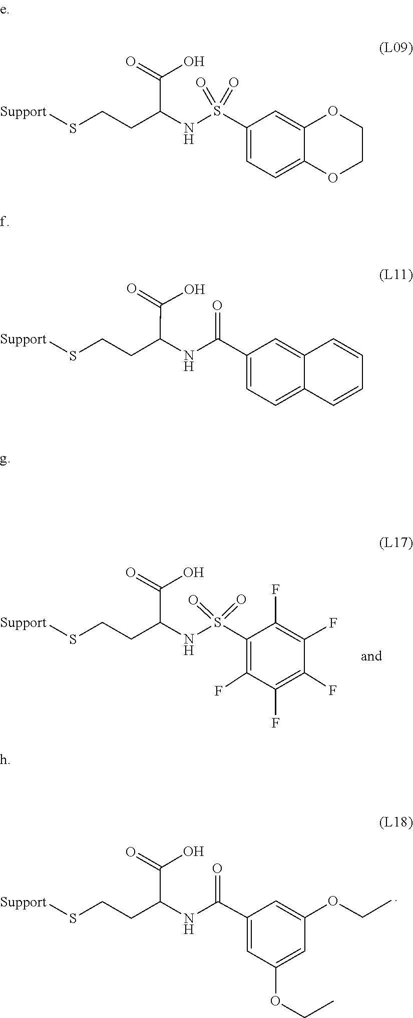

According to presently preferred embodiments, the presently disclosed chromatography ligand may be defined by a chemical structure selected from a group consisting of (a)-(f):

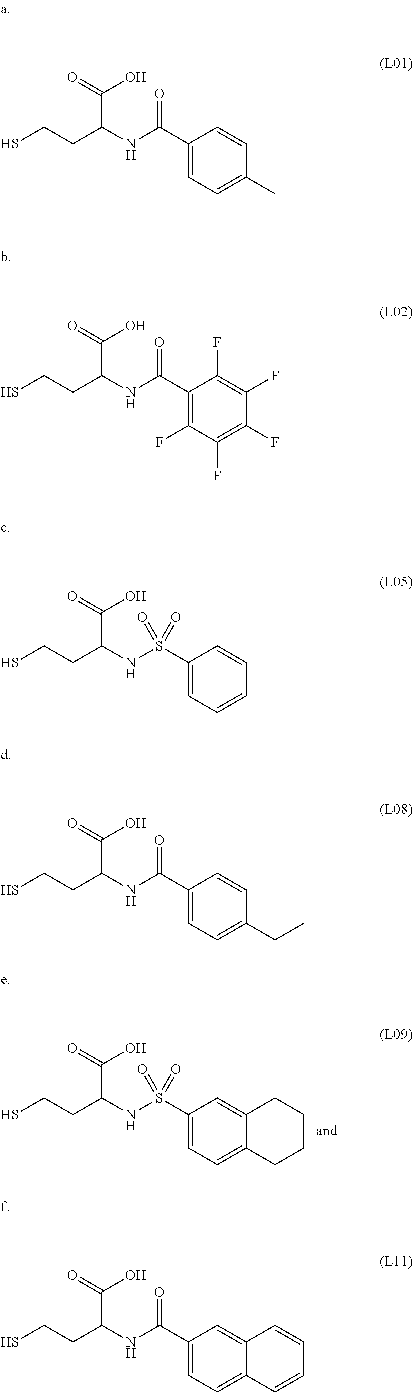 [SMILES: Cc1ccc(C(=O)NC(CCS)C(=O)O)cc1 ; O=C(NC(CCS)C(=O)O)c1c(F)c(F)c(F)c(F)c1F ; O=C(O)C(CCS)NS(=O)(=O)c1ccccc1 ; CCc1ccc(C(=O)NC(CCS)C(=O)O)cc1 ; O=C(O)C(CCS)NS(=O)(=O)c1ccc2c(c1)CCCC2 ; O=C(NC(CCS)C(=O)O)c1ccc2ccccc2c1]

When the above chromatography ligands are coupled to a support, they may be illustrated as follows:

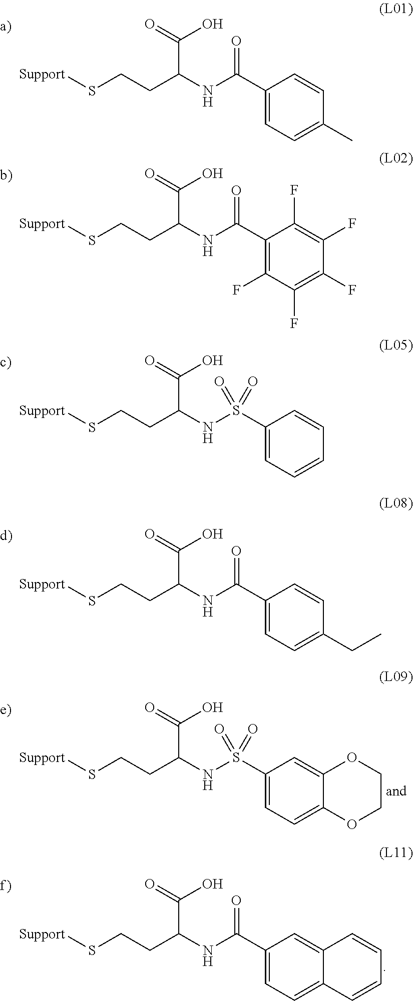

Table 1 describes the above-listed ligands (a)-(h) as well as the reference ligand, in terms of their chemical structures, predicted log S values, and lysozyme batch binding capacity.

TABLE 1
Chemical structure, predicted Log S value, and lysozyme batch binding capacity for the resin
based on the ligands of interest and reference ligand. Predicted Log S values are for the structure
with the support modeled as a methyl group. In other words, they were calculated for the Methyl-S-
ligand structures.

| | | | Lysozyme batch binding |
|---|---|---|---|
| Ligand | | | capacity* at pH 7.5 and 480 |
| no. | Chemical structure | Log S | mM NaCl |
| L00 (ref) | 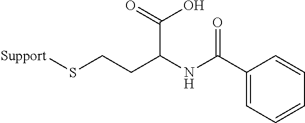 | -2,794 | 40.643 |
| L01 | 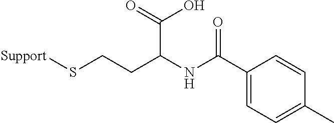 | -3,156 | 66.385 |
| L02 | 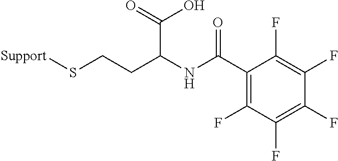 | -3,947 | 76.002 |
| L05 | 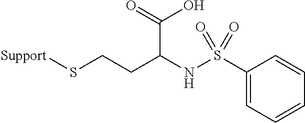 | -2,969 | 75.957 |
| L08 | 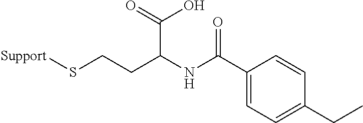 | -3,517 | 76.546 |
| L09 | 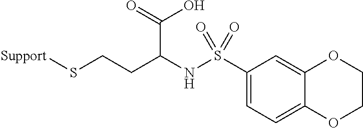 | -3,109 | 97.773 |
| L11 | 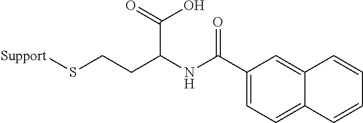 | -4,168 | 84.998 |
| L17 | 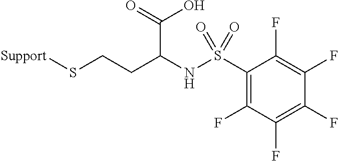 | -4,14683 | |
| L18 | 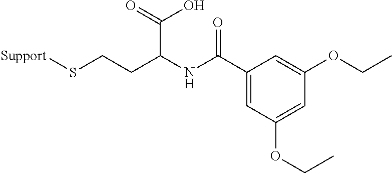 | -3,54633 | |

* Lysozyme amount bound to ligand expressed as % of lysozyme amount added to the resin.

The present disclosure further provides a method for preparing a chromatography material, comprising immobilising a plurality of the above-defined chromatography ligand to a support.

The term “separation matrix” is used herein to denote a material comprising a support to which one or more ligands comprising functional groups have been coupled. The functional groups of the ligand(s) bind compounds herein also called analytes, which are to be separated from a liquid sample and/or which are to be separated from other compounds present in the liquid sample. A separation matrix may further comprise a compound which couples the ligand(s) to the support. The terms “linker”, “extender”, and “surface extender” may be used to describe such a compound, as further described below. The term “resin” is sometimes used for a separation matrix in this field. The terms “chromatography material” and “chromatography matrix” are used herein to denote a type of separation matrix.

The term “surface” herein means all external surfaces and includes in the case of a porous support outer surfaces as well as pore surfaces.

The separation matrix may be contained in any type of separation device, as further defined elsewhere herein. As a non-limiting example, a chromatography material may be packed in a chromatography column, before adding a liquid sample to the chromatography material being contained in the chromatography column.

The herein disclosed chromatography material comprises a support to which the ligand is coupled. The term “support” has its conventional meaning in the field of bioprocessing and may alternatively be called a “support material” or a “solid phase”, which are other terms conventionally used in this field.

In formula Ia, it is illustrated the part of the ligand that is intended to couple to the support.

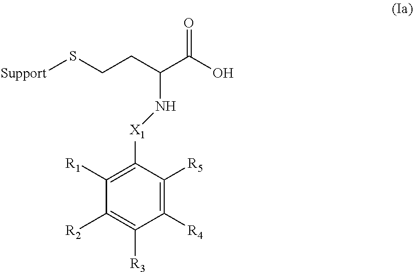

In this regard, the term “support” at the upper left end of formula Ia shows where the ligand may be coupled to the support. In case a linker or extender is used to couple the ligand to the support (as described in detail further below), such a linker or extender may also be included in the term “Support”.

There is also disclosed herein a compound defined by formula I, and as further defined when referring to a chromatography ligand.

The disclosed chromatography material comprises a support to which the ligand is coupled. The support may be made of different types of materials and may have different shapes or forms, as described in more detail below.

The support may be made from an organic or inorganic material and may be porous or non-porous. In one embodiment, the support is prepared from a native polymer, such as cross-linked carbohydrate material, e.g., agarose, agar, cellulose, dextran, chitosan, konjac, carrageenan, gellan, alginate, pectin, starch, etc. The native polymer supports are easily prepared and optionally cross-linked according to standard methods, such as inverse suspension gelation (S Hjerten: Biochim Biophys Acta 79(2), 393-398 (1964). In an especially advantageous embodiment, the support is a kind of relatively rigid but porous agarose, which is prepared by a method that enhances its flow properties, see e.g., U.S. Pat. No. 6,602,990 (Berg). In an alternative embodiment, the support is prepared from a synthetic polymer or copolymer, such as cross-linked synthetic polymers, e.g. styrene or styrene derivatives, divinylbenzene, acrylamides, acrylate esters, methacrylate esters, vinyl esters, vinyl amides etc. Such synthetic polymers are easily prepared and optionally cross-linked according to standard methods, see e.g., “Styrene based polymer supports developed by suspension polymerization” (R Arshady: Chimica e L'Industria 70(9), 70-75 (1988)). Native or synthetic polymer supports are also available from commercial sources, such as Cytiva, Sweden, for example in the form of porous particles. In yet an alternative embodiment, the support is prepared from an inorganic polymer, such as silica. Inorganic porous and non-porous supports are well known in this field and easily prepared according to standard methods.

The support of the chromatography material may be in the form of particles, such as substantially spherical, elongated or irregularly formed particles.

The Capto ImpRes base chromatography matrix (Cytiva, Uppsala, Sweden) comprises a support in the form of substantially spherical particles or beads, which have a diameter of approx. 40 μm. This is a non-limiting example of a particle suitable for inclusion of the presently disclosed ligands by coupling of the ligand to the support.

Suitable particle sizes of the presently disclosed chromatography material may be in a diameter range of 5-500 μm, such as 10-200 μm, e.g., 20-100 μm. In a specific embodiment, the average particle size is in the range of from about 20 μm to about 50 μm, such as about 20 μm, 25 μm, 30 μm, 35 μm, 40 μm, 45 μm, or 50 μm, preferably from about 25 μm to about 40 μm. Suitable average pore sizes of the presently disclosed chromatography material in the form of particles may be of any size larger than the target molecules and impurities to be separated, including but not limited to an average pore diameter of from about 9 nm (e.g., suitable for separation of monoclonal antibodies) to about 80 nm, such as about 9 nm, 10 nm, 11 nm, 12 nm, 15 nm, 20 nm, 30 nm, 50 nm, 75 nm, or 80 nm.

The skilled person in this field can easily choose the suitable particle size and porosity depending on the process to be used.

The chromatography material may be dried, such as dried particles which upon use are soaked in liquid to retain their original form. For example, such a dried chromatography material may comprise dried agarose particles.

The support of the chromatography material may alternatively be in the form of magnetic particles. The term “magnetic particle” is defined herein as a particle which is able to be attracted by a magnetic field. At the same time, magnetic particles for use in the presently disclosed method shall not aggregate in the absence of a magnetic field. In other words, the magnetic particles shall behave like superparamagnetic particles. The particle may have any symmetric shape, such as a sphere or a cube, or any asymmetric shape. Spherical magnetic particles are often called magnetic beads. It is to be understood that the terms “magnetic particle”, “magnetic bead”, “Mag particle”, “Mag bead”, “magparticle” and “magbead” may be used interchangeably herein, without limiting the scope to magnetic particles having a spherical shape. Magnetic particles suitable for use in the presently disclosed method have been described in WO2018122089, which is hereby incorporated by reference in its entirety.

The support of the chromatography material may alternatively take any other shape conventionally used in separation, such as monoliths, filters or membranes, capillaries, chips, nanofibers, surfaces, etc.

Where the support of the chromatography material comprises a monolith, a suitable average pore diameter in the monolith for the purpose of separating target molecules from impurities ranges from a minimum average pore diameter of about 9-12 nm (e.g., suitable for separation of monoclonal antibodies), and up to a maximum pore diameter of about 5 μm, such as about 0.5, 1.0, 2.0, 3.0, 4.0, or 5.0 μm.

Where the support of the chromatography material comprises nanofibers, such nanofibers may for example comprise electrospun polymer nanofibers. When in use, such nanofibers form a stationary phase comprising a plurality of pores through which a mobile phase can permeate.

The support of the chromatography material may comprise a membranous structure, such as a single membrane, a pile of membranes or a filter. The membrane may be an adsorptive membrane. Where the support of the chromatography material comprises a membranous structure, a suitable pore diameter in the membranous structure for the purpose of separating target molecules from impurities ranges from a minimum average pore diameter of about 9-12 nm (e.g., suitable for separation of monoclonal antibodies), and up to a maximum pore diameter of about 5 μm, such as about 0.5, 1.0, 2.0, 3.0, 4.0, or 5.0 μm. Where the chromatography material comprises a membranous structure, such membranous structure may for example comprise a nonwoven web of polymer nanofibers.

In the context of membranous structures, non-limiting examples of suitable polymers may be selected from polysulfones, polyamides, nylon, polyacrylic acid, polymethacrylic acid, polyacrylonitrile, polystyrene, and polyethylene oxide, and mixtures thereof.

Alternatively, the polymer may be a cellulosic polymer, such as selected from a group consisting of cellulose and a partial derivative of cellulose, particularly cellulose ester, cross-linked cellulose, grafted cellulose, or ligand-coupled cellulose. Cellulose fiber chromatography (known as Fibro chromatography; Cytiva, Sweden) is an ultrafast chromatography purification for short process times and high productivity, which utilizes the high flow rates and high capacities of cellulose fiber. Where the support of the chromatography material comprises cellulose fibers such as Fibro, a suitable pore diameter in the cellulose fiber for the purpose of separating target molecules from impurities ranges from a minimum average pore diameter of about 9-12 nm (e.g., suitable for separating monoclonal antibodies), and up to a maximum pore diameter of about 5 μm, such as about 0.1, 0.2, 0.5, 1.0, 2.0, 3.0, 4.0, or 5.0 μm.

The term “membrane chromatography” has its conventional meaning in the field of bioprocessing. In membrane chromatography there is binding of components of a fluid, for example individual molecules, associates or particles, to the surface of a solid phase in contact with the fluid. The active surface of the solid phase is accessible for molecules by convective transport. The advantage of membrane adsorbers over packed chromatography columns is their suitability for being run with much higher flow rates. This is also called convection-based chromatography. A convection-based chromatography matrix includes any matrix in which application of a hydraulic pressure difference between the inflow and outflow of the matrix forces perfusion of the matrix, achieving substantially convective transport of substance(s) into the matrix or out of the matrix, which is effected very rapidly at a high flow rate. Convection-based chromatography and membrane adsorbers are described in for example US20140296464A1, US20160288089A1, WO2018011600A1, WO2018037244A1, WO2013068741A1, WO2015052465A1, U.S. Pat. No. 7,867,784B2, hereby incorporated by reference in their entirety.

The coupling of the ligand to the support of a chromatography material as disclosed herein may be provided by introducing a linker between the support and ligand. The coupling may be carried out following any conventional covalent coupling methodology such as by use of epichlorohydrin; epibromohydrin; allyl-glycidylether; bis-epoxides such as butanedioldiglycidylether; halogen-substituted aliphatic substances such as di-chloro-propanol; and divinyl sulfone. Other non-limiting examples of suitable linkers are: polyethylene glycol (PEG) having 2-6 carbon atoms, carbohydrates having 3-6 carbon atoms, and polyalcohols having 3-6 carbon atoms. These methods are all well known in the art and easily carried out by the skilled person.

The ligand may be coupled to the support via a longer linker molecule, also known as a “surface extender”, or simply “extender”. Extenders are well known in this field, and commonly used to sterically increase the distance between ligand and support. Extenders are sometimes denoted tentacles or flexible arms. For a more detailed description of possible chemical structures, see for example U.S. Pat. No. 6,428,707, which is hereby included herein by reference. In brief, the extender may be in the form of a polymer such as a homo- or a copolymer. Hydrophilic polymeric extenders may be of synthetic origin, i.e., with a synthetic skeleton, or of biological origin, i.e., a biopolymer with a naturally occurring skeleton. Typical synthetic polymers are polyvinyl alcohols, polyacryl- and polymethacrylamides, polyvinyl ethers etc. Typical biopolymers are polysaccharides, such as starch, cellulose, dextran, agarose.

The term “eluent” is used in its conventional meaning in this field, i.e., a buffer of suitable pH and/or ionic strength to release one or more compounds from a separation matrix.

The term “eluate” is used in its conventional meaning in this field, i.e., the part(s) of a liquid sample which are eluted from a chromatography column after having loaded the liquid sample onto the chromatography column.

In the herein disclosed method for preparing a chromatography material, the density of the plurality of ligands immobilised on the support may be from about 15 to about 50 μmol/mL, such as about 15, 20, 25, 30, 35, 40, 45, or 50 μmol/mL, preferably from about 20 to about 35 μmol/mL.

Also provided by the present disclosure is a chromatography material comprising the above-defined chromatography ligand coupled to a support. The chromatography material and the support are as defined and exemplified above.

In a presently preferred embodiment, the support comprises beads having a diameter from about 25 μm to about 50 μm, preferably from about 30 μm to about 45 μm.

The herein disclosed chromatography material may have a density of the plurality of ligands, immobilised on the support, of from about 15 to about 50 μmol/mL, such as about 15, 20, 25, 30, 35, 40, 45, or 50 μmol/mL, preferably from about 20 to about 35 μmol/mL.

The chromatography material comprising a chromatography ligand as disclosed herein may be further defined by having a lysozyme batch binding capacity in the linear part of an adsorption isotherm at binding conditions pH 7.5 and 480 mM NaCl and further by that the amount of lysozyme bound to the chromatography material at equilibrium is at least 50%, such as 60% of the amount of lysozyme added to the chromatography material. The meaning of the terms “lysozyme batch binding capacity” and “linear part of an adsorption isotherm” is as defined further above.

As described elsewhere herein, the chromatography ligand may be defined by having a log S from about −2.5 to about −5.

The ligand of the chromatography material is defined by Formula I as described in detail further above.

Further provided is a use of the herein disclosed chromatography material for separating one or more target molecules from impurities.

In the context of said use, the one or more target molecules may be one or more antibodies, as described in detail elsewhere herein. Preferably, the antibodies are monoclonal antibodies. Optionally, the monoclonal antibodies are multispecific monoclonal antibodies, such as bispecific monoclonal antibodies. Alternatively, the one or more target molecules may be one or more antibody fragments. Optionally, the one or more antibody fragments may be selected from antigen-binding fragments as described and exemplified in detail elsewhere herein, e.g., Fab, Fab′, F(ab′)₂, scFv, Fv, dAb, or Fd.

Further in the context of said use, the impurities may comprise aggregates of the one or more target molecules, such as high molecular weight aggregates of the target molecules, as described in detail elsewhere herein. For example, when the target molecule is an antibody, the impurities may comprise aggregates of said antibody, such as high molecular weight aggregates of the one or more antibody fragments. Alternatively, when the target molecule is an antibody fragment, the impurities may comprise aggregates of the one or more antibody fragments, such as high molecular weight aggregates of the one or more antibody fragments.

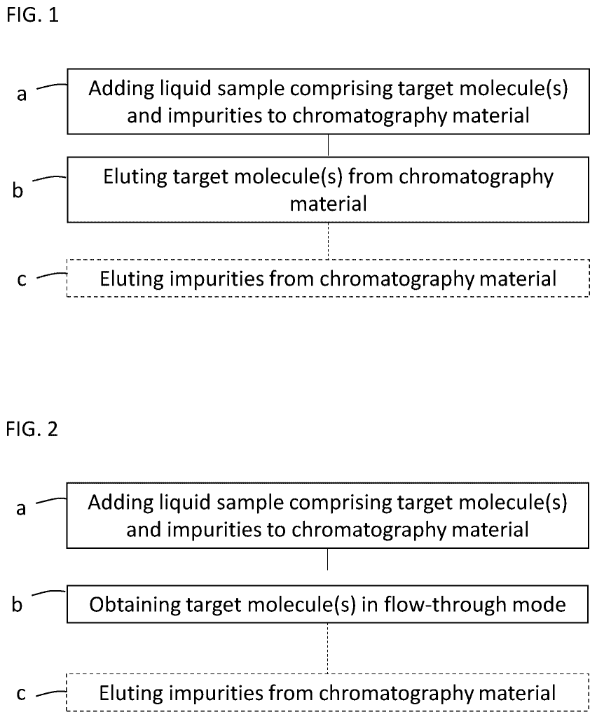

The present disclosure also solves or at least mitigates the problems associated with existing methods for separating one or more target molecules from impurities by providing, as illustrated in FIG. 1, a method for separating one or more target molecules from impurities, comprising:
a) adding a liquid sample comprising one or more target molecules and impurities to a chromatography material as disclosed herein;
b) eluting the target molecules from the chromatography material;
c) optionally eluting the impurities from the chromatography material.

In said method, the target molecules and optionally the impurities may be eluted from the chromatography material by applying an elution buffer comprising (i) a salt gradient, (ii) a pH gradient, or a combination of (i) and (ii). Elution buffers suitable for separation of various types of target molecules, e.g., monoclonal antibodies, are well known in the art and can easily be chosen by the skilled person.

It is to be understood that the term “gradient” as used in the context of elution conditions encompasses both continuous gradients and step gradients. A continuous gradient may be linear or non-linear, or a combination thereof.

The present disclosure further provides an alternative method for separating one or more target molecules from impurities, as illustrated in FIG. 2, which method comprises:
a) adding a liquid sample comprising one or more target molecules and impurities to a chromatography material as disclosed herein;
b) obtaining the target molecules in a flow-through mode, the target molecules having passed through the chromatography material essentially without binding to the chromatography material;
c) optionally eluting the impurities from the chromatography material.

The above-described flow-through method may be especially suitable when using a chromatography material comprising a support in the form of cellulosic fiber (e.g., Fibro) or nanofibers, as described in detail further above.

The above-described methods for separating target molecules from impurities, as illustrated by FIGS. 1 and 2, are based on multimodal interactions between the chromatography ligand and the molecules present in the liquid sample, i.e., electrostatic interactions, hydrophobic interactions, hydrogen bonding etc.

In the context of said methods, the one or more target molecules may be one or more antibodies, as described in detail elsewhere herein. Preferably, the antibodies are monoclonal antibodies. Optionally, the monoclonal antibodies are multispecific monoclonal antibodies, such as bispecific monoclonal antibodies. Alternatively, the one or more target molecules may be one or more antibody fragments. Optionally, the one or more antibody fragments may be selected from antigen-binding fragments as described and exemplified in detail elsewhere herein, e.g., Fab, Fab′, F(ab′)₂, scFv, Fv, dAb, or Fd.

Further in the context of said methods, the impurities may comprise aggregates of the one or more target molecules, such as high molecular weight aggregates of the target molecules, as described in detail elsewhere herein. For example, when the target molecule is an antibody, the impurities may comprise aggregates of said antibody, such as high molecular weight aggregates of the one or more antibody fragments. Alternatively, when the target molecule is an antibody fragment, the impurities may comprise aggregates of the one or more antibody fragments, such as high molecular weight aggregates of the one or more antibody fragments.

The above-disclosed methods for separating target molecules from impurities may further comprise a step (a1) preceding step (a), wherein step (a1) comprises pre-treating the liquid sample. Optionally, said pre-treating may comprise subjecting a target molecule-containing cell culture harvest to cell lysis, clarification, and/or filtration.

The above-disclosed methods for separating target molecules from impurities may further comprise a step (a2) preceding step (a), wherein step (a2) comprises pre-purifying the one or more target molecules by separating target molecules from a target molecule-containing cell culture harvest, thereby obtaining a pre-purified liquid sample comprising target molecules, before adding said pre-purified liquid sample comprising target molecules to the herein disclosed chromatography material. Optionally, said pre-purifying may comprise subjecting the target molecule-containing cell culture harvest to chromatography, or to clarification followed by chromatography.

Unless defined otherwise, all technical and scientific terms used herein have the same meaning as commonly understood by a person skilled in the art related to this invention. Also, the singular forms “a”, “an”, and “the” are meant to include plural reference unless it is stated otherwise. All publications, patent applications, patents, and other references mentioned herein are incorporated by reference in their entirety.

### Example 1

### Introduction

This Example shows experiments demonstrating a successful separation of a monoclonal antibody from Fab fragments and high molecular weight aggregates by use of the herein disclosed novel ligands. Briefly, the load contained a monomeric monoclonal antibody (mAb) of a purity of 92%, and in addition 6% of Fab fragments and 2% of high molecular weight (HMW) aggregates. The separation of mAb, Fab fragments, and HMW aggregates on chromatography material was performed in a bind and elute method. The binding was performed at favourable binding conditions of 25 mM pH 7 phosphate and elution was performed by linearly increasing NaCl concentration from low to 1M NaCl, 50 mM pH 7.5 phosphate buffer as elution buffer. In the linear salt gradient elution, first the Fab fragments (smallest in size) are eluted followed by mAb and then HMW aggregates (largest in size). Aggregates are eluted much later in the linear gradient elution indicating stronger binding affinity for ligand.

### Materials and Methods

### Materials

A liquid sample containing a monoclonal antibody (referred to as mAb1), pre-purified on a protein A chromatography column, was used in the experiments. The mAb1 has a pl value of 8.6, a molecular weight of 150 kDa and an extension coefficient of 1.58. The concentration of buffer exchanged mAb1 sample (pH 7 and conductivity 2.94 mS/cm) used for isocratic retention study was ~20 mg/ml and a monomeric purity of 98.5% and contained 1.5% HMW aggregates, and for linear salt gradient elution study was ~18 mg/ml and a monomeric purity of 92% and contained 6% of a Fab fragment and 2% of HMW aggregates. The Fab fragment was produced from mAb1 using Papain digestion method and spiked into the mAb1 sample. For high-throughput plate-based study, five model proteins, i.e., Cytochrome C, α-Lactalbumin, Lysozyme, Ovalbumin, and Human serum albumin (HSA) (Sigma-Aldrich, St. Louis, MO, USA) and the monoclonal antibody (mAb1) were used. Capto MMC ImpRes multimodal resin and the Capto ImpRes base matrix were obtained from Cytiva (Uppsala, Sweden), Chemicals including L-Homocysteine thiolactone hydrochloride was obtained from Acros Organics and acyl and sulfonyl chlorides, bromine, dichloromethane, and ethyl acetate were obtained from Sigma Aldrich. All other chemicals including sodium chloride, disodium hydrogen phosphate, sodium dihydrogen phosphate, trisodium phosphate, sodium acetate, sodium acetate trihydrate, sodium hydroxide, glacial acetic acid, blue dextran 2000 used were of analytical grade and were purchased from Merck (Darmstadt, Germany).

### Equipment and Analysis

All chromatography experiments were performed on an Äkta explorer 10 chromatographic system from Cytiva (Uppsala, Sweden) using manually packed Tricon 5/100 glass column of ~2 mL column volume (CV). The protein concentrations of eluted fractions were analysed using a SpectraMax M2^{e} spectrophotometer procured from Molecular Devices LLC (California, USA). SpectraMax 96-well plate reader were used. All gradient eluted fractions were analysed for Fab fragment, mAb1, and HMW aggregates content (%) using size exclusion chromatography high performance liquid chromatography (SEC-HPLC) using an Agilent 1200 Series HPLC procured from Agilent technologies (Santa Clara, CA, USA). A Superdex™ 200 resin column containing 13 μm particles packed in 10×300 mm high resolution column procured from Cytiva (Uppsala, Sweden) was used. The mobile phase buffer composition was 200 mM sodium phosphate at pH 6.8. The flow rate used was 0.8 mL/min with a total analysis time of 25 minutes for each analysis. Data analysis of UV 280 nm obtained by SEC-HPLC was performed using the ‘Agilent ChemStation’ software and by Äkta chromatography system using Unicorn 5.31. Titrations were performed using a Metrohm auto-titrator 905 Titrando system from Metrohm AG (Herisau, Switzerland) and analysed with the Tiamo software.

### Methods

Synthesis of Capto MMC ligand prototypes

The multimodal Capto MMC thioether ligand is derived from a thiolactone proligand. A Capto MMC ligand prototype library was synthesized via acylation or sulfonylation of the commercially available D, L-homocysteine thiolactone hydrochloride with an equimolar amount of the appropriate acyl, or sulfonyl, chloride in dichloromethane (DCM) in the presence of diisopropyl ethylamine (DIPEA). After the reaction is complete, typically 16 h, the solvent is removed in vacuo and the residue, typically a pale yellow oil, is dissolved in ethyl acetate (EtOAc) and washed sequentially in a seporatory funnel with aqueous solution of citric acid (10% w/v), aqueous solution of K₂CO₃ 10% (w/w), water, and brine. The organic phase is dried over MgSO₄, filtered, and evaporated under the reduced pressure to afford the product which is generally a white or pale yellow solid.

More specifically, the eight multimodal ligands presently disclosed were synthesized as follows:

N-toluoyl-D,L-homocysteine thiolactone (L01)-D,L-Homocyteine thiolactone hydrochloride (18.75 mmol, 2.87 g) and diisopropylethylamine (37.5 mmol, 6.53 mL) are dissolved in DCM (30 mL) and the mixture is cooled in an ice bath. Toluoyl chloride (18.75 mmol) is added slowly to the stirring solution as described in the general procedure to afford the product as white solid. ¹H NMR (CDCl₃, 300 MHz): δ=7.70 (d 2H, Ar—H), 7.24 (d, 2H, Ar—H), 6.61 (s, 1H, amide-H), 4.68 (m, 1H, thiolactone-H), 3.49-3.25 (m, 2H, thiolactone-H), 3.10 (m, 1H, thiolactone-H), 2.40 (s, 3H, Ar—CH₃), 2.03 (s, 3H, thiolactone-H).

N-pentafluorobenzoyl-D,L-homocysteine thiolactone (L02)-D,L-Homocyteine thiolactone hydrochloride (18 mmol, 2.787 g) and diisopropylethylamine (36.9 mmol, 6.492 mL) are dissolved in DCM (40 mL) and the mixture is cooled in an ice bath. Pentafluorobenzoyl chloride (18 mmol, 2.62 mL) is added slowly to the stirring solution as described in the general procedure to afford the product as white solid. ¹H NMR (CDCl₃, 300 MHz): δ=6.58 (s, 1H, amide-H), 4.63 (m, 1H, thiolactone-H), 3.40 (m, 2H, thiolactone-H), 3.13 (m, 1H, thiolactone-H), 2.07 (m, 1H, thiolactone-H).

N-benzenesulfonyl-D,L-homocysteine thiolactone (L05)-D,L-Homocyteine thiolactone hydrochloride (16 mmol, 2.478 g) and diisopropylethylamine (32.8 mmol, 5.771 mL) are dissolved in DCM (40 mL) and the mixture is cooled in an ice bath. Benzenesulfonyl chloride (16 mmol, 2.063 mL) is added slowly to the stirring solution as described in the general procedure to afford the product as white solid. ¹H NMR (CDCl₃, 300 MHz): δ=7.90 (m, 2H, Ar—H), 7.70-7.52 (d, 2H, Ar—H), 5.23 (s, 1H, amide-H), 3.78 (m, 1H, thiolactone-H), 3.32-3.23 (m, 2H, thiolactone-H), 2.86 (m, 1H, thiolactone-H), 2.07 (m, 1H, thiolactone-H).

N-(4-ethylbenzoyl)-D,L-homocysteine thiolactone (L08)-D,L-Homocyteine thiolactone hydrochloride (18.75 mmol, 2.87 g) and diisopropylethylamine (37.5 mmol, 6.53 mL) are dissolved in DCM and the mixture is cooled in an ice bath. 4-Ethylbenzoyl chloride (18.75 mmol) is added slowly to the stirring solution as described in the general procedure to afford the product as white solid (>90%). ¹H NMR (CDCl₃, 300 MHz): δ=8.35 (s, 1H, Ar), 8.00-7.80 (m, 4H, Ar), 7.68-7.49 (m, 2H, Ar), 6.74 (s, 1H, amide-H), 4.78-4.64 (m, 1H), 3.55-3.27 (m, 2H), 3.23-3.13 (m, 1H), 2.20-1.95 (m, 1H).

N-benzodioxane sulfonyl-D,L-homocysteine thiolactone (L09)-D,L-Homocyteine thiolactone hydrochloride (15 mmol, 2.323 g) and diisopropylethylamine (30 mmol, 5.28 mL) are dissolved in DCM (25 mL) and the mixture is cooled in an ice bath. 1,4-Benzodioxan-6-sulfonyl chloride (15 mmol, 3.705 g) is added slowly to the stirring solution as described in the general procedure to afford the product as white solid. ¹H NMR (CDCl₃, 300 MHz): δ=7.45-7.30 (m, 2H, Ar), 6.94 (d, 2H, Ar), 5.11 (s, 1H, amide-H), 4.30 (m, 4H, alph), 3.69 (m, 1H, thiolactone-H), 3.25 (m, 2H, thiolactone-H), 2.86 (m, 1H, thiolactone-H), 2.05 (m, 1H, thiolactone-H).

N-(2-naphthoyl)-D,L-homocysteine thiolactone (L11)-D,L-Homocyteine thiolactone hydrochloride (18.75 mmol, 2.87 g) and diisopropylethylamine (37.5 mmol, 6.53 mL) are dissolved in DCM (30 mL) and the mixture is cooled in an ice bath. 2-Naphthoyl chloride (18.75 mmol) is added slowly to the stirring solution as described in the general procedure to afford the product as white solid (92%, 4.67 g). ¹H NMR (CDCl₃, 300 MHz): δ=8.35 (s, 1H, Ar), 8.00-7.80 (m, 4H, Ar), 7.68-7.49 (m, 2H, Ar), 6.74 (s, 1H, amide-H), 4.78-4.64 (m, 1H), 3.55-3.27 (m, 2H), 3.23-3.13 (m, 1H), 2.20-1.95 (m, 1H).

N-pentafluorobenezensulfonoyl homocysteine thiolactone (L17)-D,L-homocysteine thiolactone HCl (10.02 mmol, 1.02 eq, 1.567 g), dichloromethane (20 mL), and diisopropylethylamine (2.04 eq, 20.04 mmol, 3.553 mL), are added to a Schlenk flask and flushed with N₂ and stirred until dissolved. The solution is cooled in an ice bath and pentafluorobenzenesulfonyl chloride (1 eq, 10 mmol, 2.666 g, 1.484 mL) is added dropwise. The reaction mixture is allowed to warm to rt while stirring overnight under N₂. The reaction is complete by TLC (DCM with drops of EtOH, or 1:1 EtOAc/cyclohexane). The reaction mixture is concentrated on the rotovap, then diluted with EtOAc (100 mL). This is washed with citric acid (10 wt %, 3×50 mL) then K₂CO₃ (10 wt %, 3×50 mL), and brine (2×50 mL). The organic phase is concentrated on the rotovap, and the brown solid residue (1.7 g, theoretical yield is 3.75 g) was dried on the house vac. The crude product was recrystallized from boiling EtOH/H₂O (ca. 30:10 mL) yielding light brown solid (0.896 g, 24% yield). ¹H NMR (300 MHz, CDCl₃) δ 5.72 (br, 1H, NH), 4.20 (m, 1H, CH), 3.36 (m, 2H), 2.88 (m, 1H), 2.18 (m, 1H).

N-(3,5-diethoxy-benzoyl) homocysteine thiolactone (L18)-D,L-homocysteine thiolactone HCl (10.2 mmol, 1.02 eq, 1.567 g), dichloromethane (20 mL), and diisopropylethylamine (2.04 eq, 20.4 mmol, 2.636 g, 3.55 mL), are added to a Schlenk flask and flushed with N2 and stirred until dissolved. The solution is cooled in an ice bath and the acid chloride (1 eq, 10 mmol, 2.287 g) in DCM (5 mL) is added dropwise. After 30 min, the ice bath is removed and the reaction mixture is allowed to warm to rt while stirring overnight under N2. TLC (DCM with drops of EtOH, or 1:1 EtOAc/cyclohexane) shows one spot+baseline. The reaction mixture is concentrated on the rotovap, then diluted with EtOAc (100 mL). This is washed with citric acid (10 wt %, 3×50 mL) then K₂CO₃ (10 wt %, 3×50 mL), and brine (2×50 mL). The organic phase is concentrated on the rotovap, and the white solid was dried on the house vac. Yields 2.78 g (90% yield). ¹H NMR (300 MHz, CDCl₃) δ 6.89 (d, 2H, ArH), 6.58 (t, 1H, ArH), 6.50 (br, 1H, NH), 4.64 (m, 1H, CH), 4.06 (q, 4H, O—CH₂—CH₃), 3.42 (m, 1H), 3.34 (m, 1H), 3.10 (m, 1H), 2.01 (m, 1H), 1.41 (t, 6H, —CH₃).

Following aqueous work up and crystallization where appropriate, the resulting N-acylated, or sulfonylated, thiolactone is hydrolyzed in aqueous NaOH providing the carboxylate cation exchange group, and a thiol which is then used to nucleophilically couple to the functionalized agarose base matrix (Capto ImpRes, Cytiva). Briefly, the agarose base matrix is first functionalized with terminal allyl groups using allyl glydicyl ether (AGE), which are subsequently brominated with elemental bromine. The brominated gels form epoxy termini under basic conditions which are good electrophiles to react with MMC ligand prototypes via S_{N}2 nucleophilic substitution. Multimodal ligand loadings were 25-30 μmol/mL_{Resin} as determined by titration.

### High-Throughput Plate-Based Screening Study

The binding capacity (BC) data in the linear part of the isotherm of six different proteins, i.e., Cytochrome C, α-Lactalbumin, Lysozyme, Ovalbumin, HSA, and one monoclonal antibody (mAb1), on the novel multimodal cation-exchange resins including the reference Capto MMC ImpRes resin (~25 μmol/mL_{Resin}), were obtained through high-throughput plate-based fast screening. BC data at low protein loading of 10 μg/μL_{Resin} was determined in 6 μL PreDictor™ 96-well filter plates for four different binding pH (4.5, 5.5, 6.5, 7.5) and eight different NaCl salt concentrations (0, 55, 133, 257, 480, 750, 1250, 1750 mM) using an automated liquid handling system Tecan robotic workstation procured from Tecan Group Ltd. (Mannedorf, Switzerland). Thus, the ligand prototypes were tested for 32 different binding pH and salt conditions. The binding pH 4.5 and 5.5 were based on a 25 mM acetate buffer and pH 6.5 and 7.5 based on a 50 mM phosphate buffer and the stock volumes for binding pH were calculated using a proprietary Excel application (Cytiva, Uppsala, Sweden). All protein solutions were prepared using 5 mM pH 7 phosphate buffer whereas the mAb1 load was buffer exchanged in 5 mM pH 7 phosphate in order to avoid the influence of buffer on targeted pH and salt concentration. Several experiments were repeated in duplicates.

### Estimation of System Dead Volume, Column Porosity, and Resin Particle Porosity

The external dead volume of the Äkta system was estimated by the pulse injection of 200 μL 1M NaCl in pH 6.5 10 mM phosphate buffer in the absence of column i.e. a zero dead volume connector was used in place of the column. The column porosity, ε_{C}, of manually packed columns with resin Capto MMC ImpRes coupled with different synthesized ligand prototypes was estimated by the pulse injection of 200 μL of 3 mg/ml of blue dextran 2000 (Mol. Wt. 2000 kDa) in pH 6.5 10 mM phosphate buffer into column. The column porosity, ε_{C}, was calculated using the equation:

$${ε}_{C} = \frac{{V}_{D e x} - {V}_{systemdeαd}}{{V}_{C}} \qquad ( 1 )$$

Where V_{Dex} is the column retention volume of blue dextran, V_{Systemdead} is the Äkta system dead volume, and V_{C} is the geometric column volume. The resin particle porosity, εₚ, was calculated using the equation:

ε_{T}=ε_{C}+εₚ(1−ε_{C}) (2)

Where ε_{T} is the total column porosity which was estimated from the pulse injection of 200 μL protein solution (of concentration ≈8.23 mg/mL) under non-binding condition of pH 12 100 mM Na₃PO₄ into column. The total column porosity, ε_{T}, was calculated using the equation:

$${ε}_{T} = \frac{{t}_{0} Q}{{V}_{C}} \qquad ( 3 )$$

Where t₀ is the retention time of protein under non-binding condition, Q is flow rate, and V_{C} is column volume.

### Isocratic Retention Study

Each isocratic retention experiment was performed in 5 different steps including: Step 1: Equilibration (5 CV; pH 7 25 mM phosphate buffer+X (200 mM to 1800 mM) NaCl); Step 2: Sample loading (~2 mg of protein per mL of resin; 200 μL sample load volume); Step 3: Isocratic elution (until UV is less than 2 mAU); Step 4: Strip (5 CV; pH 7.5 50 mM phosphate buffer+1000 mM NaCl); Step 5: Clean in place (CIP, 3CV) step with 1N NaOH for column regeneration and sanitization. The flow rate was set at 0.5 mL/min. The fluid effluent was monitored at 280 nm for peak detection. The retention factor, k, was calculated using the equation:

$$k = \frac{{V}_{R} - {V}_{0}}{{V}_{0}} \qquad ( 4 )$$

V_{R} is the system dead volume corrected retention volume of the protein under isocratic condition and V₀ is the column void volume and was calculated using the equation:

V₀=V_{C}ε_{C} (5)

From the isocratic retention factor, k, the total number of counter salt ions and water molecules released during mAb1 adsorption and the hydrophobic contact area (HCA) estimated by fitting the characteristic “U”-shaped curve obtained for multimodal chromatography to the preferential interaction model developed by Perkins et al. [28, 29]. The equation relating the total number of counter salt ions released and water molecules released to the retention factor, k, is given by:

$$ln k = c + \frac{( {Δν}_{+} + Δ {v}_{-} )}{g} ln {C}_{S} - \frac{n {Δν}_{1}}{mg} {C}_{S} \qquad ( 6 )$$

Where −(Δv₊+Δv₋) is the number of counter ions released during mAb1 adsorption governed by electrostatic interactions at low salt concentration and (−Δv₁) is the total number of water molecules released during adsorption of one mAb1 molecule governed by hydrophobic interactions at higher salt concentration; c is the integration constant; C_{S} is the mobile phase salt concentration; m is the molar concentration of water; n is the valency of the salt ion;

$$g = {( \frac{\partial ln {C}_{S}}{\partial ln {a}_{\pm }} )}_{T , P}$$

is the thermodynamic property of the salt ions, α is the activity of salt ions, respectively. The isocratic retention factor data was fitted to the simplified form of Eq. (6) given by:

lnk=α+βlnC_{S}−γC_{S} (7)

The model parameters β and γ are related to the (Δv₊+Δv₋) and (Δv₁) by the following expressions:

$$( {Δν}_{+} + {Δν}_{-} ) = g β \qquad ( 8 )$$

$${Δν}_{1} = \frac{gm}{n} γ \qquad ( 9 )$$

The hydrophobic contact area (HCA) during mAb1 adsorption on resin was calculated by fitting the isocratic retention factor data to the Melander's 3 parameter equation given by:

log k=A−BlogC_{S}+CC_{S} (10)

Where B and C are the electrostatic and hydrophobic interaction parameters, respectively. From the parameter, C, the HCA was calculated using the equation [31]:

$$H C A = \frac{2.3 RTC}{{σ}_{S}} \qquad ( 11 )$$

Where R is the universal gas constant, T is the absolute temperature, and σ_{S} is the molar surface tension increment of the salt.

Linear salt gradient elution study Each linear NaCl salt gradient experiment was performed in 5 different steps including:

### (i) Method1: Bind and Elute

| | | Column volumes (CVs): 1CV = 1.963 mL |
|---|---|---|
| Step | Buffer | (5 mm diameter; 10 cm length) |
| Equilibration | 25 mM pH 7 phosphate buffer | 5 CV |
| Sample loading | Buffer exchanged mAb load in | |
| | equilibration buffer:Resin load | |
| | capacity 5 g per liter of resin | |
| Column wash | With equilibration buffer | 3 CV |
| Elution | 50 mM pH 7.5 phosphate + 1M | 20CV + 10 CV @ 100% B; |
| | NaCl | 1.5 mL fractions collected |
| CIP | 1N NaOH @ 0.5 mL/min | 3 CV |
| Re-equilibration | With equilibration buffer | 8CV |

### (ii) Method2: Bind and Elute

| | | Column volumes (CVs): 1CV = 1.963 mL |
|---|---|---|
| Step | Buffer | (5 mm diameter; 10 cm length) |
| Equilibration | pH 5.5 50 mM acetate + 50 mM | 12 CV |
| | NaCl | |
| Sample loading | Buffer exchanged mAb load in | |
| | equilibration buffer:Resin load | |
| | capacity 5 g per liter of resin | |
| Column wash | With equilibration buffer | 10 CV |
| Elution | pH 8.5 25 mM acetate + 25 mM | 40CV + 10 CV @ 100% B; |
| | phosphate + 1M NaCl | 2 mL fractions collected |
| CIP | 1N NaOH @ 0.3 mL/min | 3 CV |
| Re-equilibration | With equilibration buffer | 12CV |

Elution was collected in fractions and analysed for protein concentration and for mAb1, Fab fragment, and HMW aggregate content (%) using SE-HPLC as described above. Using the SE-HPLC data, the linear gradient elution peak was de-convoluted into its components mAb1 and Fab and used to calculate the resolution, Rₛ, between mAb1 and Fab using the expression:

$${R}_{s} = 1.18 \times ( \frac{{t}_{R 2} - {t}_{R 1}}{{W}_{0.5 {h}_{1}} + {W}_{0.5 {h}_{2}}} ) \qquad ( 12 )$$

Where t_{R} is the retention time of peak and w_{0,5h} is the full width at the half maximum peak height. The retention time of mAb1 and Fab fragment peak is denoted by t_{R2} and t_{R1}, respectively, and the full width at the half maximum peak height of Fab and mAb1 is denoted by W_{0.5h}_{₁} and W_{0.5h}_{₂}, respectively.

The separation resolution for HMW aggregates was compared by plotting cumulative mAb1 yield (%) versus cumulative HMW aggregates (%). The cumulative mAb1 yield (%) and HMW aggregates (%) calculated using the SE-HPLC data using the following expressions:

$$Cumulative mAb 1 yield \% \vert i to n = \frac{Cumulative mAb 1 ( mg ) \vert i to n}{Total mAb 1 in load ( mg )} \times 100 \qquad ( 13 )$$

$$Cumulative HMW aggregate \% \vert i to n = \frac{Cumulative HMW aggregate ( mg ) \vert i to n}{Cumulative total protein in column pool ( mg ) \vert i to n} \times 100 \qquad ( 14 )$$

### Results and Discussion

The effect of ligand density, as determined by ionic capacity measurements, on the chromatographic performance was minimized by making all the prototypes with new ligands with a similar ionic capacity (20-31 μmol/mL_{Resin}) as the reference Capto MMC ImpRes resin (25 μmol/mL_{Resin}). The synthesized prototypes were tested for chromatographic performance in high-throughput plate-based studies.

Isocratic Retention of mAb1 on Capto MMC Analogue Prototypes

To understand the mAb1 adsorption behaviour on the new Capto MMC ligand prototypes, and to begin to qualitatively connect ligand structure to binding, mAb1 retention was analysed as a function of different isocratic chromatographic conditions for the new prototypes plus the reference ligand. Dimensionless isocratic retention factor, ln (k), for mAb1 vs. NaCl concentration (C_{S}), varied from 200 mM to 1800 mM at pH 7, were plotted (not shown).

The prototypes displayed a “U”-shaped dependence for the protein retention as a function of salt concentration, with a retention minimum at ca. 800 mM NaCl. The “U”-shape behaviour is a characteristic of multimodal resins, and a consequence of the contributions from ion-exchange, and other secondary interactions, such as hydrophobic interactions. For binding pH values below the pl value of mAb1 (~8.6) and lower NaCl concentrations, protein retention decreases with increasing salt concentration. As with classical CIEX chromatography, this is because buffer cations compete with the positively charged mAb for the negatively charged sites on the multimodal. However, at high NaCl concentration (≥1000 mM), protein retention increases with salt concentration, behaving like a HIC resin.

In the intermediate regime, ca. 800 to 1000 mM NaCl, there is a transition taking place in mAb1 adsorption mechanism from primarily electrostatic interactions to secondary hydrophobic interactions.

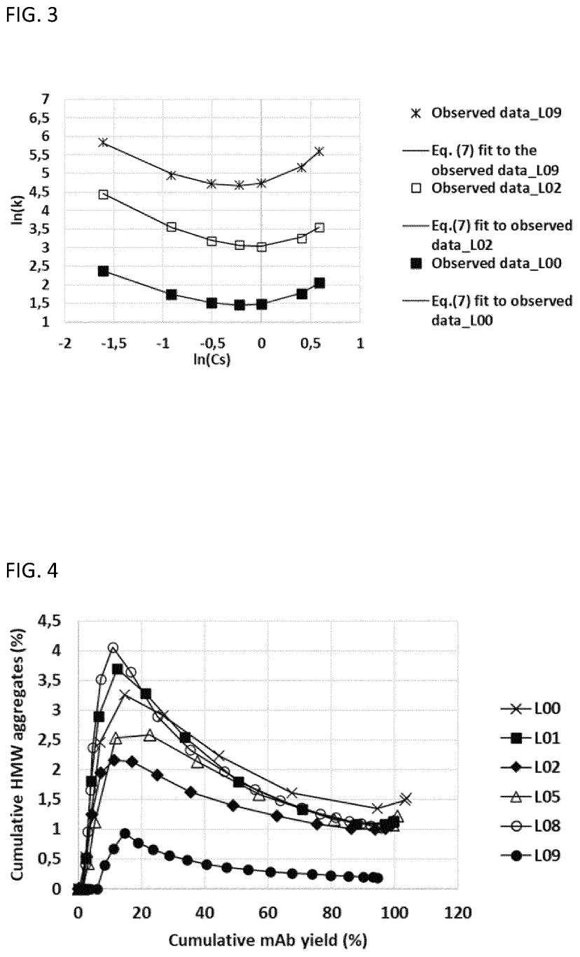

FIG. 3 displays the comparison of isocratic mAb1 retention factor, ln(k), vs. NaCl concentration varied from 200 mM to 1800 mM at pH 7 for L09, L02, and L00. More particularly, FIG. 3 shows a plot of In (k) vs. In (C_{S}) for mAb1 adsorption onto (●) L00, (x) L09, (□) L02 at pH 7. The solid lines (-) are overlapping fits to Eq. (7) and Eq. (10).

### Chromatographic Performance Using High-Throughput Binding Capacity (BC) Data

### Response Surface Analysis of High-Throughput BC Data

We set out on high-throughput plate-based studies, which provide information on protein binding to a number of targets under a variety of both pH and salt concentrations, in parallel, and are very useful for evaluating the diverse binding and elution properties of multimodal resins. These plate experiments were performed under low loading in the linear part of the isotherm, providing information about the binding behaviour and selectivity opportunities, rather than the maximum binding capacity. Again, the multimodal prototypes showed the “U”-shaped dependence of binding with salt concentration, attributed to multimodal electrostatic and other secondary interactions.

### Principal Component Analysis of BC

High-throughput plate-based binding data was collected for the prototypes with six model proteins each at 32 different pH/[NaCl] binding conditions. To gain further insights from this large amount of data, principal component analysis (PCA) was performed, yielding six principal components (PCs). Out of these 6 PCs, the first 2 PCs described a total of 85.7% of the variance in the data. The trend of PC1 score with the binding capacity of mAb1 at pH4.5, 1750 mM NaCl as depicted in a chromatographic diversity map (not shown) indicated that the score of PC1 is strongly correlated with high salt binding to mAb1. This showed that the overall conclusions from the detailed studies of mAb1 appear to be general, and are accessible from rapid, high-throughput, plate-based studies.

### High Throughput Binding Capacity of Ligand Prototypes

Table 2 shows high throughput binding capacity data in terms of % bound of specific protein at a given binding condition.

TABLE 2
High throughput binding capacity of protein (lysozyme
and monoclonal antibody, respectively) by different
ligand prototypes at different binding conditions.

| | Ligand | Lysozyme % | mAb % |
|---|---|---|---|
| | density | binding capacity | binding capacity |
| | (μmol/mL | at pH 7.5 and | at pH 5.5 and |
| Ligand no. | of resin) | 480 mM NaCl | 480 mM NaCl |
| L00 | 25 | 40.643 | 67.413 |
| L01 | 27 | 66.385 | 92.567 |
| L02 | 25 | 76.002 | 94.805 |
| L05 | 25 | 75.957 | 93.415 |
| L08 | 24 | 76.546 | 95.330 |
| L09 | 25 | 97.773 | 96.233 |
| L11 | 26 | 84.998 | 97.374 |

### Linear Salt Gradient Elution Studies

### Separation Resolution, Rₛ, Between Fab Fragment and mAb

Table 3 shows the Rₛ values for the separation resolution between Fab fragment and the monoclonal antibody (mAb1). All new ligand prototypes showed better separation resolution than the reference ligand (L00), with L09 having the highest separation resolution, followed by L02, L05, L01 and L08.

TABLE 3
Rₛ values

| Ligand | Rₛ |
|---|---|
| L00 | 0.7 |
| L01 | 0.9 |
| L02 | 1.1 |
| L05 | 1.0 |
| L08 | 0.9 |
| L09 | 1.3 |

### Separation Resolution Between HMW Aggregate and mAb

### (i) Method1: Bind and Elute

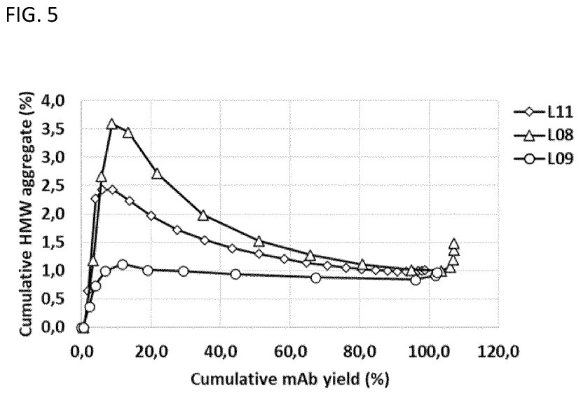

FIG. 4 shows a comparison of separation resolution between HMW aggregate and monoclonal antibody (mAb) for different ligand prototypes for the data obtained using Method1 as described further above. The load contained ~2% of HMW aggregate. The comparison of cumulative plots depicted that the novel ligands outperformed the reference ligand (L00) with L09 being the most promising one for aggregate removal.

### (ii) Method2: Bind and Elute

FIG. 5 shows a comparison of separation resolution between HMW aggregate and mAb for different ligand prototypes for the data obtained using Method2 as described further above. The load contained ~1.95% of HMW aggregates. L09 outperformed L08 and L11 for HMW aggregate removal in dual pH-salt linear gradient elution.

### CONCLUSIONS

These results indicate that the new multimodal ligand prototypes can achieve effective removal of aggregate species from challenging monoclonal antibodies and provide improved selectivity for product related impurities. It could be concluded that more hydrophobic ligands outperformed the less hydrophobic ligands. The prominent role of the secondary hydrophobic and hydrogen bonding interactions provided by the ligand chemical structure of more hydrophobic ligands in conjunction with electrostatic interactions results in their better performance with regard to removal of Fab fragment and HMW aggregate impurities.

### Example 2

An experimental design is performed with the same antibody as in Example 1, with the following variations:
(a) Testing of different ligands coupled to a support of a chromatography material, i.e., ligands comprising different hydrophobic groups.
(b) Determination of affinity of different ligands towards different multimeric forms of the antibody.

### Example 3

An experimental design is performed as in Example 1 with other target molecules than in Example 1, for example a bispecific antibody (approx. 200 kDa), or a protein not being an antibody, e.g., a protein of 50-100 kDa.

It is to be understood that the present disclosure is not restricted to the above-described exemplifying embodiments thereof and that several conceivable modifications of the present disclosure are possible within the scope of the following claims.

1. A chromatography ligand defined by formula I: 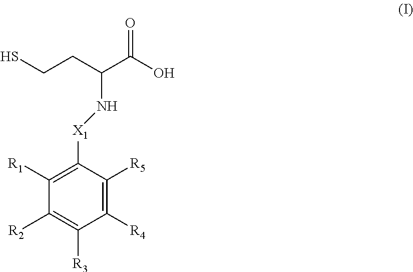
wherein:
X₁ is selected from CO and SO₂;
each of R₁-R₅ is independently selected from H, F, Cl, O, N, S, C₁₋₃ alkyl, and C₁₋₃ alkyl-X₂;
any two adjacent moieties selected from R₁-R₅ together with the atoms to which they are attached may form a 5- or 6-membered heterocyclic or carbocyclic ring; and
X₂ is selected from O, S, NH(CO), (CO)NH, NH(SO₂), and (SO₂)NH; and
provided that:
i. when each of R₁-R₅ is H, X₁ is SO₂;
ii. when any two adjacent moieties selected from R₁-R₅ together with the atoms to which they are attached form a 5-membered heterocyclic containing ring containing two oxygen atoms in the ring, X₁ is SO₂; and
iii. when three of R₁-R₅ are CH₃O, X₁ is CO.

2. The chromatography ligand according to claim 1, wherein any two adjacent moieties selected from R₁-R₅ together with the atoms to which they are attached form a 5-membered heterocyclic ring containing a maximum of one oxygen atom in the ring.

3. The chromatography ligand according to claim 1, which is defined by a chemical structure selected from a group consisting of (a)-(f):

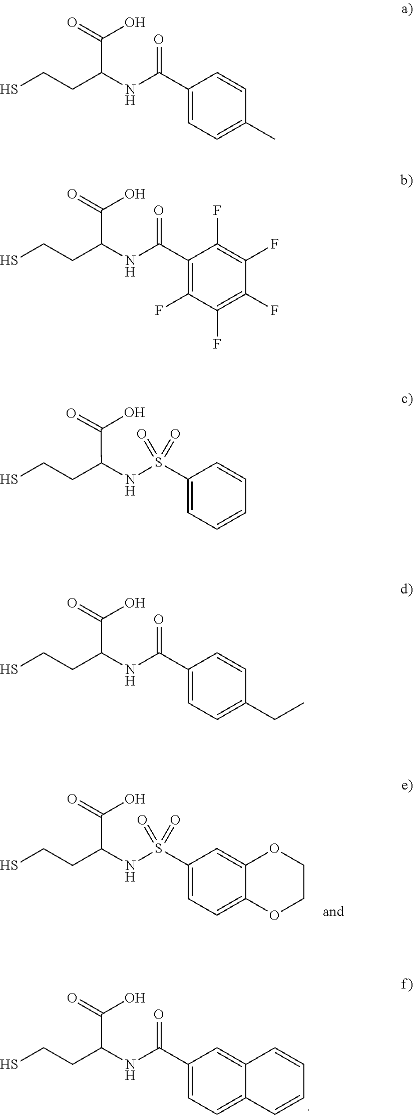 [SMILES: Cc1ccc(C(=O)NC(CCS)C(=O)O)cc1 ; O=C(NC(CCS)C(=O)O)c1c(F)c(F)c(F)c(F)c1F ; O=C(O)C(CCS)NS(=O)(=O)c1ccccc1 ; CCc1ccc(C(=O)NC(CCS)C(=O)O)cc1 ; O=C(O)C(CCS)NS(=O)(=O)c1ccc2c(c1)OCCO2 ; O=C(NC(CCS)C(=O)O)c1ccc2ccccc2c1]

4. The chromatography ligand according to claim 1, wherein said ligand, when the thiol (—SH) of formula I has been replaced with a methyl thioether (—S—CH₃), has a log S from about −2.5 to about −5.

5. A method for preparing a chromatography material, comprising immobilising a plurality of ligands according to claim 1, to a support, optionally wherein the support comprises beads having a diameter from about 25 μm to about 50 μm, preferably from about 30 μm to about 45 μm.

6. The method according to claim 5, wherein the density of the plurality of ligands immobilised on the support is from about 15 to about 50 μmol/mL, preferably from about 20 to about 35 μmol/mL.

7. A chromatography material comprising a chromatography ligand according to claim 1, coupled to a support, optionally wherein the support comprises beads having a diameter from about 25 μm to about 50 μm, preferably from about 30 μm to about 45 μm.

8. The chromatography material according to claim 7, wherein the density of the plurality of ligands coupled to the support is from about 15 to about 50 μmol/mL, preferably from about 20 to about 35 μmol/mL.

9. The chromatography material according to claim 7, wherein said material has a lysozyme batch binding capacity in the linear part of an adsorption isotherm at binding conditions pH 7.5 and 480 mM NaCl and wherein the amount of lysozyme bound to the chromatography material at equilibrium is at least 50%, such as 60% of the amount of lysozyme added to the chromatography material

10. Use of a chromatography material according to claim 7, for separating one or more target molecules from impurities.

11. The use according to claim 10, wherein the one or more target molecules are one or more antibodies, preferably wherein the antibodies are monoclonal antibodies, optionally wherein the monoclonal antibodies are multispecific monoclonal antibodies, such as bispecific monoclonal antibodies.

12. The use according to claim 11, wherein the impurities comprise aggregates of the one or more antibodies.

13. The use according to claim 12, wherein the one or more target molecules are one or more antibody fragments, optionally wherein the one or more antibody fragments are selected from antigen-binding fragments, such as Fab, Fab′, F(ab′)₂, scFv, Fv, dAb, or Fd.

14. The use according to claim 13, wherein the impurities comprise aggregates of the one or more antibody fragments.

15. A method for separating one or more target molecules from impurities, comprising:
a) adding a liquid sample comprising one or more target molecules and impurities to a chromatography material according to claim 7;
b) eluting the target molecules from the chromatography material;
c) optionally eluting the impurities from the chromatography material.

16. The method according to claim 15, wherein the target molecules and optionally the impurities are eluted from the chromatography material by applying an elution buffer comprising (i) a salt gradient, (ii) a pH gradient, or a combination of (i) and (ii).

17. A method for separating one or more target molecules from impurities, comprising:
a) adding a liquid sample comprising one or more target molecules and impurities to a chromatography material according to claim 7;
b) obtaining the target molecules in a flow-through mode, the target molecules having passed through the chromatography material essentially without binding to the chromatography material;
c) optionally eluting the impurities from the chromatography material.

18. The method according to claim 15, wherein the one or more target molecules are one or more antibodies, preferably wherein the antibodies are monoclonal antibodies, optionally wherein the monoclonal antibodies are multispecific monoclonal antibodies, such as bispecific monoclonal antibodies.

19. The method according to claim 18, wherein the impurities comprise aggregates of the one or more antibodies.

20. The method according to claim 15, wherein the one or more target molecules are one or more antibody fragments, optionally wherein the one or more antibody fragments are selected from antigen-binding fragments, such as Fab, Fab′, F(ab′)₂, scFv, Fv, dAb, or Fd.

21. The method according to claim 20, wherein the impurities comprise aggregates of the one or more antibody fragments.
